(19) United States
(12) Patent Application Publication
(10) Pub. No.: US 2002/0182238 A1
(43) Pub. Date: Dec. 5, 2002

(54) Fibers as anti-irritant agents

(75) Inventor: Isabelle Creton, Paris (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'OREAL, Paris (FR)

(21) Appl. No.: 10/101,061

(22) Filed: Mar. 20, 2002

(30) Foreign Application Priority Data
Mar. 23, 2001 (FR) 01 03956

(51) Int. Cl. A61K 7/00

(52) U.S. Cl. 424/401

(57) ABSTRACT

The invention relates to the use of fibers as anti-irritant agents, for example in a composition for topical application, and for reducing or eliminating the irritant effect of one or more irritant compounds. The invention also relates to a composition, preferably for topical application, especially a cosmetic or dermatological composition, containing fibers and one or more compounds with an irritant side effect, and to a cosmetic treatment process using the cosmetic composition according to the invention.

### BACKGROUND OF THE INVENTION

1. Field of the Invention

The present invention relates to the use of fibers as anti-irritant agents, for example in a composition for topical application, and for reducing the irritant effect of one or more compounds with an irritant effect, for example in a cosmetic composition, or for the preparation of a dermatological composition containing irritating compounds.

The invention also relates to a composition for topical application comprising one or more compounds with an irritant effect and fibers, and also to a composition for topical application containing fibers and a calmant. The invention also relates to a cosmetic treatment process using the compositions.

In the context of the present invention, the irritant effect is an irritant skin effect (or skin irritation), which may be a side effect, and it is a response from the skin usually reflected by redness, pain or stinging, this response being generated by chemical compounds of natural or synthetic origin (irritant compound), applied topically to the skin. This irritation is accompanied by an impairment in the epithelial function and/or structure, directly associated with the effect of the compound of irritant nature.

Thus, the disruptions induced by an irritant compound generally are followed by a more or less intense response from the skin, intended to restore the homeostatic equilibrium broken or to repair the damage caused. When the irritant compound has attacked the skin, it may react with certain substances pre-existing in the cells and tissues and/or may release intracellular substances. These released substances may in turn become active on other targets in the epithelium or the dermis. Thus, this is how the cascade of reactions begins, which, by recruiting blood cells and the substances they release, give rise to the irritant process which is characterized mainly by an irritation of the skin. This process is reflected especially, to varying degrees, depending mainly on the quality and/or quantity of the applied compound and/or on the user of this compound, by dysesthetic sensations (heating, sensations of burning, itching or pruritus, sensations of stinging, tautness, etc.), by redness and/or oedema.

Compounds with an irritant (side) effect, that is to say irritant compounds which can cause a skin irritation, especially for individuals (users) with sensitive skin, are present in cosmetic or dermatological compositions, obviously for other effects.

For example, cosmetic compositions containing keratolytic active agents are used to combat ageing, and especially exfoliant active agents or active agents that promote cell renewal, such as α-hydroxy acids (in particular lactic acid, glycolic acid or citric acid), β-hydroxy acids (especially salicylic acid and 5-n-octanoylsalicylic acid) and retinoids (especially all-trans or 13-cis retinoic acid, and retinol). Unfortunately, if these active agents are used in excessively large amounts, they have the drawback of causing irritation such as stinging, itching or tautness after they have been applied, which may lead to great discomfort. The use of these compounds, especially by users with sensitive skin, should thus be limited.

In addition, even certain compounds that are considered as inert in a cosmetic or dermatological composition, such as, for example, preserving agents, surfactants, fragrances, solvents or propellants, may have an irritant nature when they are applied to keratin materials and especially the skin, including the scalp, this irritant nature being dependant on the compound used and on the skin sensitivity of the user. Thus, it is common to use surfactants, emulsifiers or detergents in cosmetic or dermatological compositions, for example as emulsifiers in compositions in the form of water-in-oil (W/O) or oil-in-water (O/W) or triple emulsions, or, for example, as detergents in compositions for cleansing human skin. Similarly, it is common to incorporate preserving agents to ensure good conservation of the compositions, or of fragrances to give products a pleasant odour.

The compounds with an irritant nature are generally used in small doses. The use in small amount of these compounds may then prove to be of little benefit relative to the use of other compounds that are less active, but less or not at all irritant and thus used in larger amount, or relative to the final nature of the compound, such as, for example, the stability of the composition when it concerns emulsifiers or the correct conservation of the composition when it concerns preserving agents.

There is thus still a need, especially in the cosmetic and dermatological fields, to find a means for using these compounds without them having an irritant effect with which the user may find fault, and thus to obtain a, e.g., cosmetic or dermatological composition which, although containing compounds with an irritant (side) effect, is comfortable and non-irritant.

The inventor has now discovered that fibers make it possible to limit or even eliminate the irritant effect of these irritant compounds.

The document EP-A-1,057,477 describes a composition containing cellulose fibrils allowing to avoid the use of surfactant. By the fact of the absence of surfactant, the obtained composition is less irritant. However, this document does not disclose that fibers allow one to diminish or eliminate the irritant effect of an irritant compound present in the composition.

Thus, one subject of the present invention is the use of fibers as anti-irritant agents, especially in a cosmetic composition.

The invention also relates to the use of fibers as anti-irritant agents for the preparation of a composition for topical application intended to treat keratin materials.

The invention also relates to the use of an effective amount of fibers to reduce the irritant effect of a cosmetic composition containing one or more irritant compounds.

A subject of the present invention is also the use of an effective amount of fibers for the preparation of a dermatological composition containing one or more irritant compounds, the fibers reducing this irritant effect.

The present invention especially has the advantage of eliminating the irritation that irritant compounds could or would have caused, and also of making it possible to increase the amount of irritant compounds in cosmetic or dermatological compositions when compared with the amount normally used, for the purpose of improving the efficacy of the said compounds. Thus, surfactants, preserving agents or keratolytic active agents such as hydroxy acids or retinoids can be used without any inconvenience to the user.

Thus, the present invention also relates to a composition for topical application, especially a cosmetic or dermatological composition, characterized in that it comprises fibers and one or more irritant compounds chosen from α-hydroxy acids, β-hydroxy acids, α-keto acids, β-keto acids, anthralins, anthranoids, peroxides, minoxidil and its derivatives, lithium salts, antiperspirants, hair-removing active agents or permanent-waving active agents, depigmenting agents, capsaicin, anti-louse active agents, anionic surfactants, and mixtures thereof.

The expression “topical application” means herein an external application onto keratin materials, and “keratin materials” means the skin, the scalp, the eyelashes, the lips, the eyebrows, the nails and mucous membranes.

According to one particular embodiment of the invention, the composition contains at least two irritant compounds, for example two, three, or four irritant compounds, or more, for example two active agents and one surfactant or alternatively two active agents, one surfactant, and one solvent.

Since the composition according to the invention is preferably intended for topical application, it preferably comprises a physiologically acceptable medium, that is to say a medium that is compatible with all keratin materials such as the skin, including the scalp, the nails, mucous membranes, eyes and hair, or any other area of body skin. It may especially constitute a cosmetic or dermatological composition.

One subject of the present invention is also a process for reducing or eliminating the irritant effect of at least one irritant compound present in a composition, characterized in that an effective amount of fibers is used.

The “anti-irritant effective amount of fibers” according to the invention is an amount of fibers that is sufficient to reduce or even eliminate an irritant skin effect of the irritant compound(s) present. Thus, this amount is variable as a function of the fibers used, the amount and nature of the compound(s) with an irritant nature used and/or the sensitivity of the user to this compound. However, by way of illustration, a composition according to the invention generally comprises fibers in a concentration ranging from 0.01% to 50% by weight, preferably from 1% to 20% by weight and better still from 5% to 10% by weight relative to the total weight of the composition.

Besides having a good anti-irritant effect, the compositions containing the fibers have good cosmetic properties: comfortable when applied, texture that is easy to spread and soft.

The fibers which may be used in the composition of the invention may be hydrophilic or hydrophobic fibers, of natural or synthetic, mineral or organic origin. These fibers may be short or long, individual or organized, for example braided. They may have any shape or morphology and in particular may have a circular or polygonal (square, hexagonal or octagonal) cross section depending on the specific application envisaged. In particular, their ends can be blunted and/or polished to prevent injury. In particular, the fibers may have a length (L) ranging from 1 μm (0.001 mm) to 10 mm, preferably from 0.1 μm to 5 mm and better still from 0.1 mm to 1.5 mm, including all values and subranges between stated values such as 0.5, 0.7, 1, 1.3, 2, 4, 6 and 8 mm. Their cross section may be within a circle of diameter (D) ranging from 1 nm (0.001 μm) to 100 μm, preferably ranging from 1 nm (0.001 μm) to 50 μm and better still from 5 μm to 40 μm including all values and subranges between stated values such as 10, 20, 25, 30 and 35 μm.

Preferably, the fibers used according to the present invention have a shape factor, i.e. a ratio L/D (length/diameter) ranging from 3.5 to 2,500, better still from 5 to 500 and even better still from 5 to 150, again including all values and subranges between stated values such as 25, 50, 75 and 100.

The yarn count of the fibers is often given in denier or decitex. The denier is the weight in grams for 9 km of yarn. The fibers used according to the invention preferably have a yarn count ranging from 0.15 to 30 denier and better still from 0.18 to 18 denier, including all values and subranges between stated values such as 0.2, 0.8, 1, 3, 5, 7, 9, 11, 13, 15 and 17 denier.

The shape factor, the yarn count and the morphology of the fibers are the three factors that are important for defining a fiber.

The fibers may be those used in the manufacture of textiles and in particular silk, cotton, wool or flax fibers, cellulose fibers extracted in particular from wood, plants or algae, polyamide (Nylon®) fibers, modified cellulose (rayon, viscose or acetate, in particular rayon acetate) fibers, poly-phenyleneterephthalamide fibers, in particular Kevlaro fibers, acrylic fibers, in particular polymethyl methacrylate or poly(2-hydroxyethyl methacrylate) fibers, polyolefin fibers and in particular polyethylene or polypropylene fibers, glass, silica or aramid fibers, carbon fibers, in particular in the form of graphite, Teflon® fibers, insoluble collagen fibers, polyester, polyvinyl chloride, polyvinylidene chloride, polyvinyl alcohol, polyacrylonitrile, chitosan, polyurethane or polyethylene phthalate fibers, and fibers formed from a mixture of polymers such as those mentioned above, for instance polyamide/polyester fibers.

Examples of polyurethane fibers which may be used are poly(urethane-urea) polymer fibers, belonging to the elastane class, and especially those sold under the name Lycra® by the company DuPont.

Resorbable synthetic fibers used in surgery may also be used, for instance the fibers prepared from glycolic acid and from caprolactone (Monocryl from the company Johnson & Johnson); resorbable synthetic fibers such as the copolymer of lactic acid and of glycolic acid (Vicryl from the company Johnson & Johnson); terephthalic polyester fibers (Ethibond from the company Johnson & Johnson) and stainless steel yarns (Acier from the company Johnson & Johnson).

Mixtures of fibers may also be used.

Moreover, the fibers may or may not be surface-treated and may be coated or uncoated. They may especially be coated and/or functionalized fibers, the term “functionalized” meaning that the fibers are surface-treated so as to modify their properties.

As coated fibers which may be used in the invention, mention may be made of polyamide fibers coated with copper sulphide for an antistatic effect (for example R-STAT from the company Rhodia) or another polymer allowing a particular organization of the fibers (specific surface treatment) or a surface treatment which induces colour/hologram effects (for example Lurex fiber from the company Sildorex).

The fibers can also be functionalized, that is to say be modified so as to have a specific function. This functionalization of the fibers can be carried out both on the fibers and in the fibers and by any method which makes it possible to attach a compound to the fibers or to trap it within the cavities formed by the geometry of the fibers. Mention may be made, as methods, of, for example, coating the fibers with an active principle; fixing, to the fibers, particles enclosing an active principle, such as nanocapsules or nanospheres; adsorption in the fibers; or fixing by chemical reaction. It is thus possible to use fibers having specific functional purposes, for example fibers which are stabilized against UV radiation by modification with chemical or physical sunscreens; fibers which have been rendered bactericidal or antiseptic by modification with preservatives or antibacterials; fibers which have been coloured by modification with colouring molecules; fibers which have been rendered keratolytic or desquamating by modification with keratolytic or desquamating agents; fibers which have been rendered hydrating by modification with hydrating agents or water-retaining polymers; fibers which have been rendered fragrant by modification with a fragrance; fibers which have been rendered analgesic or soothing by modification with an antiinflammatory or a soothing agent; or fibers which have been rendered resistant to perspiration by modification with an antiperspirant.

According to their properties, the fibers used according to the present invention may be introduced into an aqueous medium, an oily medium or into a powder.

The fibers which may be used according to the invention are preferably chosen from polyamide fibers, poly-p-phenyleneterephthalamide fibers and cotton fibers, and mixtures thereof. Their length may range from 0.1 to 10 mm and preferably from 0.1 to 1 mm, their mean diameter may range from 5 to 50 μm and the shape factor preferably ranges from 5 to 150.

In particular, the polyamide fibers sold by Etablissements P. Bonte under the name Polyamide 0.9 dtex 0.3 mm, having a mean diameter of from 15 to 20 μm, a yarn count of about 0.9 dtex (0.81 denier) and a length ranging from 0.3 mm to 1.5 mm, may be used. Poly-p-phenyleneterephthalamide fibers with a mean diameter of 12 μm and a length of about 1.5 mm may also be used, such as those sold under the name Kevlar Floc by the company Du Pont Fibers. These polyamide fibers are preferably introduced into an oily medium or introduced via a dry route into a powder.

Cotton fibers with a mean diameter of 20 μm, a length of 0.3 mm and a shape factor of 15 may also be used, such as those sold by the company Filature de Lomme, by the company Textiles des Dunes, by the Institut Textile de France or by the company Velifil.

As indicated above, the fibers may be present in the composition according to the invention in an amount ranging, for example, from 0.01% to 50% by weight, preferably from 1% to 20% by weight and better still from 5% to 10% by weight of active material relative to the total weight of the composition.

The irritant compounds, which are liable to cause a skin irritation, especially for individuals with sensitive skin, include active agents, preserving agents, surfactants, fragrances, solvents or propellants, and mixtures thereof, including a mixture of irritant compounds which mixture provides an irritant (side) effect even where one or even all irritant compounds are individually present in below-irritant amounts. In the invention the irritant compound(s) are present in an irritant amount, meaning an amount sufficient to cause irritation in the absence of the invention fibers.

As active agents liable to have an irritant side effect, mention may be made, for example, of:

keratolytic agents such as α-hydroxy acids, for instance glycolic acid, lactic acid, malic acid, citric acid, tartaric acid and mandelic acid, and derivatives thereof; β-hydroxy acids, for instance salicylic acid and its derivatives; α-keto acids, for instance ascorbic acid or vitamin C and its derivatives; β-keto acids; retinoids, for instance retinol and its esters, retinal, retinoic acid and its derivatives (such as those described in documents FR-A-2 570 30 377, EP-A-199 636, EP-A-325 540 and EP-A-402 072);

anthralins (dioxyanthranol);

anthranoids (for example those described in document EP-A-319 028);

peroxides (especially benzoyl peroxide);

minoxidil and its derivatives such as minoxidil sulfates and minoxidil carbamates;

lithium salts;

hair dyes or hair colorants, for instance paraphenylenediamine (p-PDA) and certain derivatives thereof such as N-phenyl-p-PDA and toluene2,5-diamine sulphate; meta-phenylenediamine (m-PDA) and certain derivatives thereof such as toluene-3,4-diamine; ortho-phenylenediamine (o-PDA);

fragrancing alcoholic solutions (fragrances, eaux de toilette, after-shaves and deodorants);

antiperspirants (certain aluminium salts);

hair-removing active agents or permanent-waving active agents (thiols, aqueous ammonia);

depigmenting agents (hydroquinone);

capsaicin;

anti-louse active agents (pyrethrin);

detergents (ionic and nonionic);

and mixtures thereof.

Among the salicylic acid derivatives that may be mentioned more particularly are 5-n-octanoylsalicylic acid and 5-n-dodecanoylsalicylic acid, and esters thereof.

Preserving agents that may be mentioned include phenoxyethanol, and bactericides such as octoxyglycerol or 1-(2-ethylhexyl)glyceryl ether (Sensiva SC 50 sold by the company SEPPIC), and caprylyl-glycol (or 1,2-octanediol) sold by the company Straetmans.

Surfactants which may be mentioned more particularly include anionic surfactants such as alkyl sulphates and alkyl ether sulphates, for instance lauryl sulphate and lauryl ether sulphate, and salts thereof, especially sodium salts thereof.

The composition may contain one or more compounds with an irritant nature, which may have the same activity or different activity, for example a surfactant, an active agent and a preserving agent, or a preserving agent and a surfactant.

Preferably, the amount of the irritant compound(s) in the composition of the invention is sufficient to cause a skin irritation, the fibers making it possible to attenuate or even eliminate this irritation. This irritant amount depends on the compound(s) used and on the final nature of the composition containing it (them). It may range, for example, from 0.0001% to 50% by weight, preferably from 0.01% to 30% by weight and better still from 0.1% to 15% by weight relative to the total weight of the composition.

The physiologically acceptable medium for the compositions for topical application according to the invention may more particularly contain water and optionally a physiologically acceptable organic solvent chosen, for example, from lower alcohols containing from 1 to 8 carbon atoms and preferably 1 to 6 carbon atoms, for instance ethanol, isopropanol, propanol or butanol; polyethylene glycols containing from 6 to 80 ethylene oxides; polyols, for instance propylene glycol, isoprene glycol, butylene glycol, glycerol or sorbitol.

This medium may also be an anhydrous medium, especially an oily medium containing oils and/or fatty substances other than oils.

When the physiologically acceptable medium is aqueous, it preferably has a pH that is compatible with the skin, preferably ranging from 3 to 8 and better still from 4.5 to 7.

When the composition comprises an aqueous or aqueous-alcoholic medium, it is possible to add a fatty phase (or oily phase) to this medium, so that the compositions of the invention are softer and more nourishing.

The oily phase usually contains at least one oil. As oils that may be used in the composition of the invention, mention may be made, for example, of:

hydrocarbon-based oils of animal origin, such as perhydrosqualene;

hydrocarbon-based oils of plant origin, such as liquid triglycerides of fatty acids containing from 4 to 10 carbon atoms, for instance heptanoic or octanoic acid triglyceride or alternatively, for example, sunflower oil, corn oil, soybean oil, marrow oil, grape seed oil, sesame oil, hazelnut oil, apricot oil, macadamia oil, arara oil, sunflower oil, castor oil, avocado oil, caprylic/capric acid triglycerides for instance those sold by the company Stearineries Dubois or those sold under the names Miglyol 810, 812 and 818 by the company Dynamit Nobel, jojoba oil or karite butter oil;

synthetic esters and synthetic ethers, especially of fatty acids, for instance oils of formulae R¹COOR² and R¹OR² in which R¹ represents a fatty acid residue containing from 8 to 29 carbon atoms and R² represents a branched or unbranched hydrocarbon-based chain containing from 3 to 30 carbon atoms, such as, for example, purcellin oil, isononyl isononanoate, isopropyl myristate, 2-ethylhexyl palmitate, 2-octyldodecyl stearate, 2-octyldodecyl erucate or isostearyl isostearate; hydroxylated esters such as isostearyl lactate, octyl hydroxystearate, octyldodecyl hydroxystearate, diisostearyl malate, triisocetyl citrate and fatty alkyl heptanoates, octanoates and decanoates; polyol esters, for instance propylene glycol dioctanoate, neopentyl glycol diheptanoate and diethylene glycol diisononanoate; and pentaerythritol esters, for instance pentaerythrityl tetraisostearate;

linear or branched hydrocarbons of mineral or synthetic origin, such as volatile or non-volatile liquid paraffins, and derivatives thereof, petroleum jelly, polydecenes, and hydrogenated polyisobutene such as parleam oil;

fatty alcohols containing from 8 to 26 carbon atoms, for instance cetyl alcohol, stearyl alcohol and a mixture thereof (cetylstearyl alcohol), octyldodecanol, 2-butyloctanol, 2-hexyldecanol, 2-undecylpentadecanol, oleyl alcohol or linoleyl alcohol;

alkoylated and especially ethoxylated fatty alcohols such as oleth-12;

partially hydrocarbon-based and/or silicone-based fluoro oils, for instance those described in document JP-A-2 295 912. Examples of fluoro oils which may also be mentioned include perfluoromethylcyclopentane and perfluoro-1,3-dimethylcyclohexane, sold under the names “Flutec PC1®” and “Flutec PC3®” by the company BNFL Fluorochemicals; perfluoro-1,2-dimethylcyclobutane; perfluoroalkanes such as dodecafluoropentane and tetradecafluorohexane, sold under the names “PF 5050®” and “PF 5060®” by the company 3M, or alternatively bromoperfluorooctyl sold under the name “Foralkyl®” by the company Atochem; nonafluoromethoxybutane sold under the name “MSX 4518®” by the company 3M and nonafluoroethoxyisobutane; perfluoromorpholine derivatives, such as the 4-trifluoromethylperfluoromorpholine sold under the name “PF 5052®” by the company 3M;

silicone oils, for instance volatile or non-volatile polymethylsiloxanes (PDMSs) containing a linear or cyclic silicone chain, that are liquid or pasty at room temperature, especially cyclopolydimethylsiloxanes (cyclomethicones) such as cyclohexasiloxane; polydimethylsiloxanes comprising alkyl, alkoxy or phenyl groups, that are pendent or at the end of a silicone chain, these groups containing from 2 to 24 carbon atoms; phenylsilicones, for instance phenyltrimethicones, phenyldimethicones, phenyltrimethylsiloxydiphenylsiloxanes, diphenyldimethicones, diphenylmethyldiphenyltrisiloxanes, 2-phenylethyltrimethylsiloxysilicates and polymethylphenylsiloxanes;

mixtures thereof.

In the list of oils mentioned above, the expression “hydrocarbon-based oil” means any oil mainly comprising carbon and hydrogen atoms, and optionally ester, ether, fluoro, carboxylic acid and/or alcohol groups.

Other fatty substances that may be present in the oily phase include, for example, fatty acids containing from 8 to 30 carbon atoms, for instance stearic acid, lauric acid, palmitic acid and oleic acid; waxes, for instance lanolin, beeswax, carnauba wax or candelilla wax, paraffin wax, lignite wax or microcrystalline waxes, ceresin or ozokerite, synthetic waxes such as polyethylene waxes, Fischer-Tropsch waxes; gums such as silicone gums (dimethiconol); silicone resins such as trifluoromethyl-Cl-4-alkyldimethicone and trifluoropropyldimethicone; and silicone elastomers, for instance the products sold under the names “KSG” by the company Shin-Etsu, under the names “Trefil”, “BY29” or “EPSX” by the company Dow Coming or under the names “Gransil” by the company Grant Industries.

These fatty substances may be chosen in a varied manner by a person skilled in the art so as to prepare a composition having the desired properties, for example in terms of consistency or texture.

The compositions according to the invention may be in any form, including forms conventionally used for topical application, and especially in the form of aqueous, aqueous-alcoholic or oily solutions, oil-in-water (O/W) or water-in-oil (W/O) or multiple emulsions, aqueous or oily gels, liquid, pasty or solid anhydrous products, or dispersions of a fatty phase in an aqueous phase with the aid of spherules, these spherules possibly being polymer nanoparticles such as nanospheres and nanocapsules, or lipid vesicles of ionic and/or nonionic type. These compositions can be prepared according to the usual methods.

In addition, the compounds used according to the invention may be more or less fluid and may have the appearance of a white or coloured cream, an ointment, a milk, a lotion, a serum, a paste or a mousse. They may optionally be applied to the skin in the form of an aerosol. They may also be in solid form, and for example in the form of a stick.

According to one particular embodiment of the invention, the composition according to the invention is a water-in-oil (W/O) or oil-in-water (O/W) emulsion. The proportion of the oily phase in the emulsion may range from 5% to 80% by weight and preferably from 5% to 50% by weight relative to the total weight of the composition. The oils, emulsifiers and co-emulsifiers used in the composition in emulsion form may be chosen from those conventionally used in cosmetics or dermatology. The emulsifier and the co-emulsifier are generally present in the composition in a proportion ranging from 0.3% to 30% by weight and preferably from 0.5% to 20% by weight relative to the total weight of the composition. The emulsion may also contain lipid vesicles.

The emulsions generally contain at least one emulsifier chosen from amphoteric, anionic, cationic and nonionic emulsifiers, used alone or as a mixture. The emulsifiers are chosen in a suitable manner depending on the emulsion to be obtained (W/O or O/W emulsion).

As emulsifying surfactants that may be used for the preparation of the W/O emulsions, mention may be made, for example, of alkyl esters or ethers of sorbitan, of glycerol or of sugars; silicone surfactants, for instance dimethicone copolyols such as the mixture of cyclomethicone and of dimethicone copolyol sold under the name “DC 5225 C” by the company Dow Coming, and alkyldimethicone copolyols, such as the laurylmethicone copolyol sold under the name “Dow Corning 5200 Formulation Aid” by the company Dow Corning and the cetyl dimethicone copolyol sold under the name Abil EM 90® by the company Goldschmidt.

For the O/W emulsions, examples of emulsifiers which may be mentioned include nonionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alkyl ethers; sugar esters, for instance sucrose stearate.

According to one particular embodiment of the invention, the fibers used according to the invention are combined with one or more calmant active agents chosen from the other anti-irritant agents and/or antiinflammatories, these calmants reinforcing the antiirritant effect of the fibers.

Thus, a subject of the invention is also a composition for topical application, containing, in a physiologically acceptable medium, fibers and at least one calmant chosen from allantoin; beta-glycyrrhetinic acid and extracts and complexes containing it; planktons, escin and plant extracts containing it; xanthine derivatives; waters and extracts of flowers and plants; asiatic acid and plant extracts containing it; bisabolol; fruit extracts; algae; pyrrolidone carboxylates; oils of plant origin; essential oils; acexamic acid and transexamic acid; ursolic acid and extracts containing it; polysaccharides containing fucose; electrolytes; galactolipids; amino acids, derivatives thereof and salts thereof; TNF-alpha antagonists; substance P antagonists; and mixtures thereof.

As above-mentioned, the calmants used according to the invention include:

allantoin;

β-glycyrrhetinic acid, extracts containing it such as, for example, extract of Glycyrrhiza glabra (liquorice) and complexes containing it, such as the allantoin/glycyrrhetinic acid complex;

lyophilized or non-lyophilized planktons, extracts thereof and complexes thereof;

escin and plant extracts containing it, for instance extract of common horse-chestnut;

xanthine derivatives, for instance diethylaminoethyl-theophylline hydrochloride;

waters and extracts (for example aqueous, aqueousalcoholic or aqueous-glycolic extracts) of flowers and plants, for instance cornflower water, camomile water, mint water, lime water, rose water, extracts of peony, extracts of hawthorn, extracts of yarrow, extracts of mallow, extracts of marigold, extracts of sweet clover, extracts of sage, elder water, extracts of ginkgo biloba, extracts of arnica, extracts of oregano, extracts of green tea, extracts of waterlily flowers, extracts of iris and extracts of birch bark;

asiatic acid and plant extracts containing it, for instance Centella asiatica;

bisabolol;

fruit extracts, for instance extract of pineapple or extract of papaya;

algae, especially of the type such as Laminaria (for example red or brown algae);

pyrrolidone carboxylates and especially of zinc (Zn-PCA) or of copper (Cu-PCA);

oils of plant origin, for instance canola seed oil and karite oil;

essential oils, for example of coriander, of balm, of lavender, of mint and of camomile, and mixtures thereof;

acexamic acid and transexamic acid (trans-4-amino-methylcyclohexanecarboxylic acid);

ursolic acid and extracts containing it, for example extract of rosemary leaf;

polysaccharides containing fucose, for instance Fucogel 1000, sold by Solabia (aqueous solution comprising 1% polysaccharide solids comprising fucose, galactose and galacturonic acid);

electrolytes and in particular an aqueous mixture comprising from 30% to 35% magnesium chloride, from 20% to 28% potassium chloride, from 3% to 10% sodium chloride, from 0.2% to 1% calcium chloride, from 0.1% to 0.6% magnesium bromide and from 0.1% to 0.5% of insolubles, the said mixture being referred to herein as “Dead Sea bath salts” since it corresponds to the main salts contained in the Dead Sea;

galactolipids obtained, for example, from oat, such as, for example, digalactosyl diglyceride or monogalactosyl diglyceride;

amino acids, derivatives thereof and salts thereof, such as the sodium salt of amino acids grafted onto cocoyl chains, sold in the form of a mixture under the name Sepicalm S by the company SEPPIC, capryloylglycine sold under the name Lipacide C8G by the company SEPPIC and the mixture of capryloylglycine, cinnamon and sarcosine sold under the name Sepicontrol A5 by the company SEPPIC;

TNF-alpha antagonists such as lisophylline, A802715, sulfasalazine, CDP-571 (anti-TNF-alpha antibody) and MDL-201112;

substance P antagonists such as sendide, spantide II, the peptides described in document EP-A-680 749, and the extracts of filamentous bacteria described in document EP-A-761 204; and

mixtures thereof.

The amount of calmant(s) may range, for example, from 0.001% to 20% by weight and preferably from 0.01% to 15% by weight relative to the total weight of the composition.

In a known manner, the composition of the invention may also contain adjuvants that are common in cosmetics or dermatology, such as hydrophilic or lipophilic gelling agents, hydrophilic or lipophilic active agents other than those mentioned above, preserving agents, antioxidants, solvents, fragrances, fillers, lipophilic or hydrophilic sunscreeens, bactericides, odour absorbers, dyestuffs, salts and polymers (for example acrylate/Dimethicone copolymer sold under the name KP-561 by Shin-Etsu, as dispersant). The amounts of these various adjuvants are those conventionally used in the field under consideration, and, for example, from 0.01% to 20% relative to the total weight of the composition. Depending on their nature, these adjuvants may be introduced into the fatty phase, into the aqueous phase and/or into lipid spherules.

As fillers which may be used in the composition of the invention, mention may be made, for example, besides pigments, of silica powder; talc; polyamide particles and especially those sold under the name Orgasol by the company Atochem; polyethylene powders; microspheres based on acrylic copolymers, such as those based on ethylene glycol dimethacrylate/lauryl methacrylate copolymer sold by the company Dow Corning under the name Polytrap; expanded powders such as hollow microspheres and especially the microspheres sold under the name Expancel by the company Kemanord Plast or under the name Micropearl F 80 ED by the company Matsumoto; powders of natural organic materials such as crosslinked or noncrosslinked corn starch, wheat starch or rice starch, such as the powders of starch crosslinked with octenyl succinate anhydride, sold under the name Dry-Flo by the company National Starch; silicone resin microbeads such as those sold under the name Tospearl by the company Toshiba Silicone; and mixtures thereof. These fillers may be present in amounts ranging from 0% to 20% by weight and preferably from 1% to 10% by weight relative to the total weight of the composition.

The composition used according to the invention may contain one or more UV screening agents (or sunscreens) which may be a chemical screening agent or a physical sunblock or a mixture of such screening agents.

Examples of UV screening agents which may be mentioned, for example, include the octyl methoxycinnamate sold especially by the company Hoffmann-Laroche under the name Parsol MCX.

The composition according to the invention is suitable, depending on the active agents contained in this composition, in particular for body and hair hygiene and especially for treating acne-prone skin, for regrowth of the hair, for preventing hair loss, for combating the greasy appearance of the skin or the hair, protecting against the harmful aspects of sunlight or in the treatment of physiologically dry skin, for preventing and/or combating light-induced or chronological ageing, and for making up the skin, the eyelashes and mucous membranes (the lips). Thus, when it contains anti-ageing active agents, it may be intended for combating ageing of the skin, and in particular for combating wrinkles and/or fine lines in the skin, and for giving a smooth complexion. When it contains surfactants and especially detergent surfactants, it may be intended for cleansing and/or removing make-up from the skin, the hair or any other keratin material.

The composition of the invention may thus constitute a care or treatment composition for the skin (including the scalp), keratin fibers (hair, eyelashes or eyebrows), the nails or the lips, or an antisun or artificial tanning composition, or alternatively a product for cleansing or removing make-up from the skin, the hair, the eyebrows or the eyelashes, a deodorant product or a fragrancing compound. It is then generally uncoloured or faintly coloured, and it may optionally contain cosmetic or dermatological active agents. It may then be used as a care base for the skin or the lips (lip balms, for protecting the lips against cold and/or sunlight and/or wind), as a day or night care cream for facial skin and/or body skin. It may also be in the form of a medicated or unmedicated, colouring or non-colouring shampoo, and of a conditioning compound.

The composition according to the invention may also constitute a coloured cosmetic composition and especially a make-up composition for the skin, keratin fibers (hair or eyelashes) and/or mucous membranes, in particular a foundation, a blusher, a face powder, an eyeshadow, a mascara, an eyeliner, a concealer compound in stick form, a nail varnish, a lipstick or a lip gloss, optionally having care or treatment properties, or a body tattoo.

Thus, a subject of the invention is also the use of the composition as defined above, to care for, treat, cleanse and/or make up keratin materials (skin including the scalp, keratin fibers and mucous membranes).

The present invention also relates to a treatment process, characterized in that it uses the composition according to the invention.

Preferably, the cosmetic treatment process consists in applying a composition as described above to the skin, the scalp and/or mucous membranes.

The examples which follow serve to illustrate the invention without, however, being limiting in nature. The names are, depending on the case, the chemical names or CTFA names (International Cosmetic Ingredient Dictionary and Handbook) and the amounts are given as percentage by weight, except where otherwise mentioned.

### EXAMPLE 1

### O/W Emulsion

| Phase A1 | |
|---|---|
| Glycerol | 7% |
| EDTA (sodium salt) | 0.05% |
| Phenoxyethanol/parabens (preserving agents) | 1% |
| Water | qs 100% |
| Phase A2 | |
| Salicylic acid | 2% |
| Triethanolamine | 2.05% |
| Water | 7% |
| Phase B1 | |
| Cetearyl alcohol | 1.2% |
| Oleth-12 | 0.3% |
| Stearyl alcohol | 1% |
| Glyceryl stearate/PEG 100 stearate | 2.5% |
| (Arlacel 165) | |
| Hydrogenated polyisobutene | 3% |
| Octyl methoxycinnamate (Parsol MCX) | 5% |
| Acrylate/dimethicone copolymer (KP-561) | 0.6% |
| (dispersant) | |
| Phase B2 | |
| Fragrance | 0.5% |
| Cyclopentasiloxane | 7% |
| Octoxyglycerol (Sensiva SC50) | 0.5% |
| Phase C | |
| Sepigel 305 (SEPPIC) | 0.7% |
| Phase D | |
| Polyamide fibers (Nylon-66) | 8% |
| Phase E | |
| Modified starch (Dry Flo) | 3% |

Procedure: phases A1 and A2, preheated to 80° C., are mixed together and phase B1, preheated to 80° C., is introduced into the mixture with stirring. A fluid emulsion is obtained, which is cooled to 60° C., followed by introducing phase B2 and then phase D and, after cooling to 40° C., phase E, with stirring.

A non-irritant white cream capable of treating greasy skin is obtained.

### EXAMPLE 2

### O/W Emulsion

| Phase A | |
|---|---|
| Glycerol | 7% |
| EDTA (sodium salt) | 0.05% |
| Phenoxyethanol | 1% |
| Water | qs 100% |
| Phase B1 | |
| Cetearyl alcohol | 1.2% |
| Oleth-12 | 0.3% |
| Stearyl alcohol | 1% |
| Glyceryl stearate/PEG 100 stearate | 2.5% |
| (Arlacel 165) | |
| Hydrogenated polyisobutene | 3% |
| Octyl methoxycinnamate (Parsol MCX) | 5% |
| Acrylate/dimethicone copolymer (KP-561) | 0.6% |
| (dispersant) | |
| Phase B2 | |
| Fragrance | 0.5% |
| Cyclopentasiloxane | 7% |
| Phase C | |
| Sepigel 305 (SEPPIC) | 0.7% |
| Phase D | |
| Capryloylglycine | 0.5% |
| Water | 2% |
| Phase E | |
| Polyamide fibers (Nylon-66) | 8% |

The procedure is identical to that of Example 1.

A non-irritant white cream capable of treating greasy skin is obtained.

Comparative example: a composition identical to that of Example 2 but without fibers was prepared. A test was performed on a panel of 5 individuals, consisting in applying the composition of Example 2 and that of the comparative example in a half-face test (simultaneous application of the two compositions, one to one half of the face, and the other to the second half of the face), the application being performed in the form of a mask in a proportion of 10 mg/cm² of composition. This test showed that for all the individuals on the panel, the composition of Example 2 was much more comfortable than that of the comparative example (none or virtually no stinging with the composition according to the invention and a lot of stinging with the composition of the comparative example).

French patent application 0103956 is incorporated herein by reference, as are all documents, articles, reports, standards and patents referred to above.

The invention also relates to a method of providing a non-irritating composition by adding to a composition containing an irritant amount of an irritant compound an anti-irritant amount of fibers, and by combining, mixing, contacting, etc. (“mixing”) irritant compounds and fibers, in irritant amounts and anti-irritant amounts, respectively, in the same composition.

Finally, the invention relates to an article of manufacture comprising the invention fibers and, associated therewith in the form of instructions, packaging, insert, etc. indicia indicating the anti-irritant effect and/or use of the invention fibers.

1. A method for preparing a non-irritating composition, comprising mixing an irritant amount of at least one irritant compound and an anti-irritating effective amount of fibers.

2. The method of claim 1, wherein said composition is a composition suitable for topical application to keratin materials.

3. The method of claim 1, wherein said composition is in the form of an emulsion.

4. The method of claim 1, wherein said composition is a dermatological or cosmetic composition.

5. The method of claim 1, wherein the fibers have a length (L) ranging from 1 μm to 10 mm.

6. The method of claim 1, wherein the fibers have a cross section which is within a circle of diameter (D) ranging from 1 nm to 100 μm.

7. The method of claim 1, wherein the fibers have a shape factor (L/D) ranging from 5 to 150.

8. The method of claim 1, wherein the fibers have a yarn count ranging from 0.15 to 30 denier.

9. The method of claim 1, wherein the fibers are selected from the group consisting of silk, cotton, wool, flax fibers, cellulose fibers, polyamide fibers, modified cellulose fibers, poly-p-phenyleneterephthalamide fibers, acrylic fibers, polyolefin fibers, glass, silica, aramid fibers, carbon fibers, Teflon® fibers, insoluble collagen fibers, polyester fibers, polyvinyl chloride fibers, polyvinylidene chloride fibers, polyvinyl alcohol fibers, polyacrylonitrile fibers, chitosan fibers, polyurethane fibers, polyethylene phthalate fibers, fibers formed from a mixture of polymers, resorbable synthetic fibers, and mixtures thereof.

10. The method of claim 1, wherein the fibers are coated and/or functionalized.

11. The method of claim 1, wherein the fibers are selected from the group consisting of polyamide fibers, poly-p-phenyleneterephthalamide fibers, cotton fibers, and mixtures thereof.

12. The method of claim 1, wherein the fibers are mixed in an amount ranging from 0.01% to 50% by weight relative to the total weight of the composition.

13. The method of claim 1, wherein the irritant compound is selected from the group consisting of active agents, preserving agents, surfactants, fragrances, solvents, propellants, and mixtures thereof.

14. The method of claim 1, wherein the irritant compound is selected from the group consisting of α-hydroxy acids, β-hydroxy acids, α-keto acids, β-keto acids, retinoids, anthralins, anthranoids, peroxides, minoxidil and its derivatives, lithium salts, hair dyes, hair colorants, fragrancing alcoholic solutions, antiperspirants, hair-removing active agents, permanent-waving active agents, depigmenting agents, capsaicin, anti-louse active agents, detergents, bactericides, anionic surfactants, and mixtures thereof.

15. The method of claim 1, wherein the irritant compound is selected from the group consisting of glycolic acid, lactic acid, malic acid, citric acid, tartaric acid, mandelic acid and derivatives thereof, salicylic acid and its derivatives, 5-n-octanoylsalicylic acid, esters thereof, 5-n-dodecanoylsalicylic acid, esters thereof, retinol and its esters, retinal, retinoic acid and its derivatives, phenoxyethanol, octoxyglycerol, caprylyl glycol, alkyl sulphates, salts thereof, alkyl ether sulphates, salts thereof, and mixtures thereof.

16. The method of claim 1, wherein the amount of irritant compound(s) ranges from 0.0001% to 50% by weight relative to the total weight of the composition.

17. The method of claim 1, wherein the composition further comprises a physiologically acceptable medium.

18. A composition for topical application, comprising an anti-irritant effective amount of fibers and an irritant amount of one or more irritant compounds selected from the group consisting of α-hydroxy acids, β-hydroxy acids, α-keto acids, β-keto acids, anthralins, anthranoids, peroxides, minoxidil and derivatives thereof, lithium salts, antiperspirants, hair-removing active agents, permanent-waving active agents, depigmenting agents, capsaicin, anti-louse active agents, anionic surfactants, and mixtures thereof.

19. The composition according to claim 18, wherein said irritant compound is selected from the group consisting of glycolic acid, lactic acid, malic acid, citric acid, tartaric acid, mandelic acid, salicylic acid, ascorbic acid, alkyl sulphates, alkyl ether sulphates, salts of alkyl sulphates, salts of alkyl ether sulphates, and mixtures thereof.

20. The composition according to claim 18, comprising at least one of 5-n-octanoylsalicylic acid and 5-n-dodecanoylsalicylic acid.

21. The composition according to claim 18, comprising at least two irritant compounds.

22. The composition according to claim 18, wherein the amount of irritant compound ranges from 0.0001% to 50% by weight relative to the total weight of the composition.

23. A composition for topical application, comprising a physiologically acceptable medium, fibers, and at least one calmant selected from the group consisting of allantoin; beta-glycyrrhetinic acid and extracts and complexes containing it; planktons, escin and plant extracts containing it; xanthine derivatives; waters and extracts of flowers and plants; asiatic acid and plant extracts containing it; bisabolol; fruit extracts; algae; pyrrolidone carboxylates; oils of plant origin; essential oils; acexamic acid and transexamic acid; ursolic acid and extracts containing it; polysaccharides containing fucose; electrolytes; galactolipids; amino acids, TNF-alpha antagonists; substance P antagonists; and mixtures thereof.

24. The composition according to claim 23, wherein the amount of calmant(s) ranges from 0.001% to 20% by weight relative to the total weight of the composition.

25. The composition according to claim 23, further comprising at least one irritant compound.

26. The composition according to claim 25, wherein the irritant compound is selected from the group consisting of α-hydroxy acids, hydroxy acids, α-keto acids, β-keto acids, retinoids, anthralins, anthranoids, peroxides, minoxidil, lithium salts, hair dyes, hair colorants, fragrancing alcoholic solutions, antiperspirants, hair-removing active agents, permanent-waving active agents, depigmenting agents, capsaicin, anti-louse active agents, detergents, bactericides, anionic surfactants, and mixtures thereof.

27. The composition according to claim 25, wherein the fibers are present in an anti-irritant effective amount.

28. The composition according to claim 27, wherein the fibers are present in an amount ranging from 0.01% to 50% by weight relative to the total weight of the composition.

29. The composition according to claim 18, wherein the composition is in the form of an emulsion.

30. The composition according to claim 18, wherein said composition is a care, treatment and/or make-up composition for keratin materials.

31. A treatment process, comprising applying the composition of claim 18 to keratin.

32. A treatment process, comprising applying the composition of claim 23 to keratin.
